(19) United States
(12) Patent Application Publication
(10) Pub. No.: US 2004/0249373 A1
(43) Pub. Date: Dec. 9, 2004

(54) Needle electrode

(76) Inventors: Dietrich H.W Gronemeyer, Sprockhovel (DE); Huseyin Sahinbas, Bochum (DE); Andreas Bracke, Bochum (DE); Martin Deli, Mulheim (DE); Marion Denk, Bochum (DE); Katja Gonschorek, Bochum (DE); Jorn Richter, Munster (DE); Jurgen Speder, Bochum (DE)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/481,190

(22) PCT Filed: Jun. 10, 2002
(86) PCT No.: PCT/EP02/06345

(30) Foreign Application Priority Data
Jun. 21, 2001 (DE) 101 29 912.5

(51) Int. Cl. A61B 18/14

(52) U.S. Cl. 606/41; 606/49

(57) ABSTRACT

Needle electrode for therapy, especially percutaneous galvanotherapy of tumors, which can be visualized by image-generating procedures, which is provided with a coating of platinum and/or an insulating material, as well as a process for the manufacture of the needle electrode according to the invention.

The Invention relates to a needle electrode for therapy, especially percutaneous galvanotherapy of tumors, which is suitable for visualization by image-generating procedures. The invention relates further to a process for the manufacture of the needle electrode according to the invention.

Today various therapies are available for the treatment of primary tumors, skin tumors or metastases. Here the following therapies may be mentioned: surgical removal of the tumor, cryotherapy, hyperthermia, chemotherapy, alcoholic ablation, radio frequency ablation or electrochemical therapy.

Electrochemical tumor therapy (ECT) is also known as galvanotherapy. This method is primarily used to treat tumors which are inoperable for functional or esthetic reasons, can no longer be treated with radiotherapy or have developed resistances to chemotherapy. Electrochemical therapy (ECT) or galvanotherapy consists in placing electrodes on the tumorous tissue, such as skin metastases, lymph node metastases or isolated organ metastases, and passing OC electricity through the tumorous tissue. A high enough overall amount of electricity leads to the destruction or even necrosis (complete death) of the tumorous tissue.

As soon as DC electricity is applied to the electrodes, the pH value and the electric charge of the tumor tissue changes due to various chemoelectrical processes. The electric field thus built up in the area of tumor causes charged particles to migrate within the electric field.

Negatively charged particles (anions) move towards the positively charged electrode (anode), while positively charged particles (cations) move towards the cathode (negative electrode). In this process, which is known as charge separation or dissociation larger charged particles like proteins are also separated according to their charge. An important factor in the destruction of the tumor cells is the polarizing change that takes place in the cell membranes and significantly disturbs the metabolic functions of the cell membranes (electrolyte pumps, nutrient pumps, etc.). The specific equilibrium of the cancer cells, which is indispensable for important life processes, is thus disrupted, causing the cells to die.

This treatment method is increasingly used in oncology, as the electrical resistance of tumorous tissue is significantly lower than that of healthy tissue. The electricity flow is concentrated essentially on the harmful tissue, which permits selective destruction of the malignant (harmful) tissue. The destroyed tumor tissue is degraded, eliminated and replaced with scar tissue by natural processes, e.g. through increased eating cell activity.

An extended form of this electrochemical therapy is its combination with chemotherapy. The destructive effect of DC electricity on the tumor tissue can be enhanced by additionally introducing cytostatics (chemotherapeutic substances), such as mitomycin, adriblatin, epirubicin and cisplatin, into the tumor. These cytostatics are mostly cationic substances that move from the anode in the electric field through the tumor tissue towards the cathode. In this manner, cytostatics are introduced Into, and distributed within, the tumor tissue selectively and in concentrated form, so that they produce an optimum effect in systemic chemotherapy or local cytostatic perfusion without electrotherapy, the introduction of the substances is not always controllable, so that healthy tissue may be destroyed as well.

A further effect is the change that takes place in the cell membrane potential due to the electric field. This change causes the cells to open so that the absorption of cytostatics is more effective than it would otherwise be. The acidic conditions in the electric field caused by the anode lead to increased cytostatics activity. As a result, the, effectiveness coefficient is many times higher.

For this reason, electrodes designed as thin needles—or as cannulas for combined electrotherapy/chemotherapy—are used for electrotherapeutic treatment. Conventional needles or cannulas are made of copper or stainless steel, which may or may not be alloyed with copper. A major disadvantage of these needles lies in the fact that the copper alloy is subject to electrochemical decomposition (galvanic corrosion). Where copper is present, the copper ions formed are toxic to the organism in high concentrations. Moreover, the conductivity and the resistance of the needle/cannula decrease. The electric field is thus not built up in an optimum manner, which adversely impacts the treatment conditions. Negative effects on the tumor tissue and the healthy tissue due to interaction between the cytostatics introduced and the copper ions cannot be completely ruled out.

Of interest are also needles—especially those made of medical steel—that are used as electrodes in the electrocorrosive detachment/deposition of implants in the vascular area. Such electrodes are mostly applied in the neck/back area and often lead to painful and unsightly superficial burns or scars.

Therefore, in selecting materials for needle electrodes and cannulas, it is important to take account of physical properties (conductivity, resistance, strength) on the one hand and of the risk of rejection and tissue Inflammation (compatibility) on the other hand.

In view of these requirements, the objective of the invention is to provide a needle electrode that is not only electrically conductive and highly resistant to the conditions induced as the electric field builds up, but also widely compatible (biocompatible) and inert to cytostatics. Furthermore, such a needle electrode should not leave any major superficial burns and/or scars at the place of application. It would also be desirable to have a process for the manufacture of needle electrodes with the above properties.

To meet this objective, the invention suggests, based on a needle electrode of the type mentioned at the outset, an electrode that is coated with platinum and/or an insulating polymer layer, especially a needle electrode with a platinum-coated titanium body.

In medicine, titanium is used in the manufacture of bone nails, prostheses, needles, etc. due to its properties that are biocompatible with the human organism and its excellent shock and impact resistance. Moreover, titanium is an ideal material for needle electrodes or cannulas an account of its physical properties—i.e. very good electric conductivity. However, given its corrosion and pitting potential, titanium or its alloys is/are today rarely used as electrode material.

For improved corrosion resistance, a gives titanium body/object can be provided with a passivating, oxidizing coating. However, that solution is not satisfactory for electrotherapy.

For this reason, the invention suggests that the titanium body be coated with platinum. Platinum belongs to the group of noble metals that show little electrochemical corrosion. Platinum electrodes are known to be good electrodes, as they have good electric conductivity and are highly resistant. Applying a platinum coating to the titanium body increases the needle electrode's corrosion and pitting resistance, while leaving its high electric conductivity unaffected. Given the high price of platinum, making needle electrodes of 100% platinum would be financially unwise in view of the resultant high treatment costs. Furthermore, the use of platinum or platinum alloys for the electrode body must be ruled out, as platinum is a very soft material. Strength is a major requirement for needle electrodes that are introduced into In the human body.

Studies have shown that coating a titanium body with noble metals is a very difficult process. Noble metal layers rarely adhere permanently to the titanium body. They tend to come off or dissolve within a very short time. For electrotherapy, the titanium body needs to be bonded to the platinum layer permanently or at least for the duration of the treatment. This requirement is met by the PVD process.

For this reason, an appropriate coating is a platinum coating that Is applied using the PVD process (Physical Vapour Deposition). There are three different technologies. In a preferred embodiment, platinum is vaporized in a vacuum chamber, ionized and accelerated and then deposited onto the titanium body. Due to the high acceleration of the ions applied; a thin platinum layer adheres relatively durably to the titanium body.

Other technologies, such as the atomization/noble gas plasma technology, the ion beam removal technology or combinations of these technologies such as plasma-assisted metallizing or ion implating can be used for platinum coating as well. [Lit.: Römpp, Chemie Lexikon, Thieme Vedag, 9, erweiterte und neubear-beitete Auflage]

To guarantee corrosion resistance and sufficiently durable adhesion of the platinum layer to the needle electrode, the thickness of the platinum layer is between 0.1 micron and 3.0 microns, the preferred thickness being approx. 1.0 micron. The diameter of the titanium body is between 0.1 mm and 1.0 mm, preferably 0.5 to 0.8 mm. Surprisingly enough, it was found that the corrosion resistance of the needle electrode is dependent on the ratio of the titanium body diameter to the platinum layer thickness. This ratio is between 1 to 0.00075 and 1 to 0.0025 (diameter of titanium body to platinum layer). Thin titanium bodies are preferably provided with a thicker layer in relative terms in order to guarantee corrosion resistance.

The ratio of 1 to 0.00125 has proved to be especially appropriate.

The needle electrode according to the invention is suitable for visualization by Image-generating systems, In particular core spin (resonance) tomography, computer tomography and ultrasonic visualization. During the treatment, visualization of the tumor and the needle electrodes is indispensable. The needle electrodes are introduced into the tumor through the skin and the body tissue. The interface between tumorous tissue and non-tumorous tissue must be clearly visible to prevent healthy cells from being destroyed and it must further be possible to see the exact position of the needle.

The preferred length of the needle electrode is between 3 and 20 cm, more preferably between 6 and 14 cm, which allows both skin metastases and soft tissue tumors to be treated.

A further preferred embodiment of the invention is a needle electrode covered with a non-conductive, insulating polymer layer, especially a platinum-coated needle electrode of titanium. Notably in the case of deep tumors rather than skin metastases, the needle electrodes are introduced percutaneously and guided down into the tumor. The percutaneous Introduction length depends on the location of the tumor. Normally, healthy cells are located along the introduction length. When voltage is applied, the healthy cells in this area are irritated.

The insulating needle electrode does not harm healthy tissue along the introduction length. The voltage is applied exclusively to the tumor, which means that the electric field with its destructive effect is built up only in the, tumor. For this reason, the insulating layer is so designed that the tip, or rather a defined length at the end of the needle electrode, is not coated. The defined length depends on how far the needle electrode projects into the tumor. This in turn depends on the size of the tumor. The defined length up to the needle tip is here is generally defined as needle tip area. If an electrode is to be provided with a platinum coating and an insulating coating the platinum coating, may be confined to the needle tip area, with a certain amount of overlap between the coatings being desirable.

Conventional stainless steel needles/cannulas have no insulating layers; they often leave burn marks at the introduction point.

For this reason, the insulating coating according to the invention can be used also for other medical instruments that are employed for electrolytic or electrochemical treatment, especially for electrolytic detachment of occlusion coils as used in endovascular or endovasal treatment of vascular aneurysms, for example. The preferred electrodes for this purpose are stainless steel electrodes of medial steel with insulating coatings, but platinum-coated titanium electrodes can be used as well.

The thickness of the coating depends on the materials and procedures used and must be such that good adhesion is ensured and the electrode is safely insulated in the coated area.

The polymer used is parylene N, preferably parylene D and more preferably parylene C. These polymers have excellent dielectric properties and are Ideal barrier plastics. The monomer Is polymerized and deposited on the needle using the CVD process (Chemical Vapour Deposition). The CVD method is based on the Gorham process.

A further embodiment of the invention is the insulating coating of PTFE (polytetrafluorethylene). This type of coating is preferably applied in a spray operation.

The polymer layer thickness is between 0.01 mm and 0.09 mm, preferably between 0.025 mm and 0.05 mm.

The advantages of this Insulating coating are: reduced friction in dry condition, electric insulation and the very thin, transparent layer.

According to the invention, the needle electrode is designed as a cannula for electro-chemo-therapy. Electro-chemo-therapy is the combination of is galvanotherapy and chemotherapy.

Furthermore, the invention relates to the manufacture of needle electrodes according to the invention for use in electrotherapy, especially for percutaneous galvanotherapy of tumors, wherein the titanium body of the needle electrode is coated with platinum using a PVD process.

The PVC process comprises in particular the following process steps:

Platinum metal vaporization and ionization In a vacuum chamber,

Addition of reactive gases—optional,

Application of electric current,

Acceleration of the ions formed onto the titanium body and deposition of same on said body.

This process permits the titanium body to be coated with platinum in an Ideal manner, The addition of reactive gases helps to form the actual layer material which precipitates onto the titanium body located some distance away.

Using the deposition process as described by Gorham, a non-conductive polymer is applied to the needle. For the purpose of depositing the coating, the parylene polymers are precipitated from the gas phase (Gorham process). First the solid dimer di-para-xylylene is vaporized at approx. 150° C. At approx. 680° C. the dimer is quantitatively broken at the two methylenemethylene links, which leads to the formation of stable monomeric p-xylylene. Subsequently the monomer polymerizes at room temperature on the titanium body in the deposition chamber.

A further embodiment of the invention is the insulating coating of PTFE (polytetrafluorethylene). This type of coating is preferably applied in a spray operation.

Below is a detailed description of the invention based on studies and drawings.

### EXAMPLE 1

### Tests Relating to the Corrosion and Pitting Resistance of Needle Electrodes with Different Coatings

The corrosion and pitting resistance of various gold and platinum-coated ECT titanium needles was determined by introducing two needles each into a pig liver at equal spacing and applying DC electricity to them. Tests of different durations were conducted.

| | | Layer | Electrode | | | Titanium |
|---|---|---|---|---|---|---|
| | | Thickness | Spacing | DC | Time | Body |
| | Coating | [microns] | [mm] | [mA] | [min:s] | Diameter |
| 1. | Au | / | 25 | 80 | 10 | 0.8 mm dia. |
| 2. | Pt | 1 micron | 25 | 80 | 10 | 0.8 mm dia. |
| 3. | Pt | 1 micron | 25 | 80 | 20 | 0.5 mm dia. |
| 4. | Aurobond, | / | 25 | 80 | 20 | 0.8 mm dia. |
| | tem- | | | | | |
| | pered + Pt | | | | | |
| 5. | Flash | 0.2 microns | 25 | 80 | 20 | 0.5 mm dia. |
| | Gold | | | | | |

Result:

The test has revealed that the platinum coatings (2 and 3)—unlike the gold coatings—are scarcely affected by corrosion and pitting. The gold coatings showed a change in the surface structure after only a short time.

In the case of the smaller platinum-coated titanium body (0.5 mm dia.), minor dissolution occurred after 20 minutes. This was effectively countered by increasing the layer thickness.

1. Needle electrode for therapy, especially percutaneous galvanotherapy of tumors, which can be visualized by image-generating procedures, characterized in that it is provided with a coating consisting of platinum and/or an insulating polymer.

2. Needle electrode to claim 1, characterized in that it has a platinum-coated titanium body.

3. Needle electrode to claim 2, characterized in that the platinum coating is a PVD coating.

4. Needle electrode to any of claims 2 or 3, characterized in that the thickness of the platinum coating is between 0.1 micron and 3.0 microns, the preferred thickness being approx. 1.0 micron.

5. Needle electrode to any of claims 2 to 4, characterized in that the is diameter of the titanium body is between 0.1 mm and 1.0 mm, preferably between 0.5 mm and 0.8 mm.

6. Needle electrode to any of the above claims, characterized in that the needle electrode, with the exception of the needle tip area, is covered with an electrically non-conductive, insulating polymer.

7. Needle electrode to claim 6, characterized in that the polymer used is parylene N, parylene D or preferably parylene C.

8. Needle electrode to claim 6, characterized in that the insulating coating consists of polytetrafluorethylene (PTFE).

9. Needle electrode to any of claims 6 to 8, characterized in that the layer thickness of the polymer is between 0.001 mm and 0.09 mm, preferably between 0.0025 mm and 0.05 mm.

10. Needle electrode to any of the above claims, characterized in that it is designed as a cannula for electro-chemo-therapy.

11. Needle electrode to any of the above claims, characterized in that its body is made of medical steel.

12. Needle electrode to claim 1, characterized in that it measures 3 to 20 cm in length, preferably 6 to 14 cm.

13. Process for the manufacture of a needle electrode for electrotherapy, especially for percutaneous galvanotherapy of tumors, characterized in that the titanium needle electrode is coated with platinum using the PVD process.

14. Process to claim 13, characterized in that the PVD process comprises the following process stages:
Platinum metal vaporization and ionization in a vacuum chamber,
Addition of reactive gases—optional,
Application of electric current,
Acceleration of the ions formed onto the titanium body and deposition of same on said body.

15. Process to claims 13 or 14, characterized in that the electrode, except for the area of the electrode tip, is additionally coated with a non-conductive polymer.

16. Process to claim 15, characterized in that the polymer used is parylene N, preferably parylene D, and more preferably parylene C.

17. Process to any of claims 15 or 16, characterized in that the non-conductive polymer is applied using the Gorham deposition process.

18. Process to claim 16, characterized in that the insulating coating is applied in a spray operation.

19. Process to claim 18, characterized in that the material used for the coating is polytetrafluorethylene (PFTE).
